(19) United States
(12) Patent Application Publication
(10) Pub. No.: US 2010/0005711 A1
(43) Pub. Date: Jan. 14, 2010

(54) Lighted Algae Cultivation Systems

(75) Inventor: Clayton V. McNeff, Andover, MN (US)

Correspondence Address: PAULY, DEVRIES SMITH & DEFFNER, L.L.C., Plaza VII-Suite 3000, 45 South Seventh Street, MINNEAPOLIS, MN, 55402-1630, US

(73) Assignee: SARTEC CORPORATION, Anoka, MN (US)

(21) Appl. No.: 12/499,344

(22) Filed: Jul. 8, 2009

Related U.S. Application Data
(60) Provisional application No. 61/079,155, filed on Jul. 9, 2008.

(51) Int. Cl. A01H 13/00; A01G 7/00; F21S 4/00

(52) U.S. Cl. 47/1.4; 362/249.02

(57) ABSTRACT

Embodiments of the present invention relate to algae cultivation systems that include light sources and related methods. In an embodiment, the invention includes an algae cultivation system including a tank configured to hold a liquid medium and a rotor disposed within the tank. The rotor can be configured to be rotated while disposed within the tank. The system can also include a light source coupled to the rotor such that the light source rotates within the tank when the rotor rotates within the tank. Other embodiments are also described herein.

This application claims the benefit of U.S. Provisional Application No. 61/079,155, filed Jul. 9, 2008, the contents of which are herein incorporated by reference.

### FIELD OF THE INVENTION

The present invention relates to algae cultivation systems that include light sources and related methods.

### BACKGROUND OF THE INVENTION

Significant resources have been devoted to developing systems to reduce the amount of carbon dioxide in the atmosphere. The various strategies pursued can be grouped into two broad categories: reduction of carbon emissions and capture of atmospheric carbon.

In nature, plants efficiently capture atmospheric carbon through the process of photosynthesis. Using sunlight as energy, plants convert carbon dioxide and water into the precursors of carbohydrates and other plant constituents. Many different types of plants and microorganisms capture considerable amounts of carbon dioxide. Algae are photosynthetic organisms that occur in most habitats. They vary from small, single-celled forms to complex multicellular forms. Algae are estimated to generate as much as 80 percent of the Earth's oxygen. It is also estimated that algae fix 90 gigatons of carbon per year.

Various attempts have been made at designing algae culture systems in order to capture carbon dioxide. In general, there are two types of algae culture systems: open culture systems and closed culture systems. Open culture systems are open to the atmosphere. They have the advantage of being relatively inexpensive to construct. However, open culture systems are subject to atmospheric temperature fluctuations, are susceptible to contamination issues, and suffer substantial losses of water due to evaporation. In contrast, closed culture systems are closed to the atmosphere and therefore provide the advantages of a controlled environment, lower evaporative water loss, and fewer contamination issues. However, many closed culture systems require relatively complex structures and therefore have substantially higher construction and operating costs. In addition, many closed culture systems have issues associated with insufficient light penetration, algae growth on walls that can be difficult to clean, and poor temperature control.

For at least these reasons, a need remains for algae cultivation systems and methods.

### SUMMARY OF THE INVENTION

Embodiments of the present invention relate to algae cultivation systems that include light sources and related methods. In an embodiment, the invention includes an algae cultivation system including a tank configured to hold a liquid medium and a rotor disposed within the tank. The rotor can be configured to be rotated while disposed within the tank. The system can also include a light source coupled to the rotor such that the light source rotates within the tank when the rotor rotates within the tank.

In an embodiment, the invention includes a method of making an algae cultivation system. The method can include mounting a light source on a rotor and positioning the rotor within a tank configured to hold a liquid medium.

In an embodiment, the invention includes a method of operating an algae cultivation system. The method can include filling a tank with a liquid medium including algae and rotating a light source mounted on a rotor within the tank.

The above summary of the present invention is not intended to describe each discussed embodiment of the present invention. This is the purpose of the figures and the detailed description that follows.

### BRIEF DESCRIPTION OF THE FIGURES

The invention may be more completely understood in connection with the following drawings, in which:

FIG. 1 is a schematic view of an algae cultivation system in accordance with an embodiment of the invention.

FIG. 2 is a schematic view of an algae cultivation system in accordance with another embodiment of the invention.

FIG. 3 is a schematic view of an algae cultivation system in accordance with another embodiment of the invention.

FIG. 4 is a schematic view of an algae cultivation system in accordance with another embodiment of the invention.

FIG. 5 is a schematic view of a rotor for an algae cultivation system in accordance with an embodiment of the invention.

FIG. 6 is a schematic view of a rotor for an algae cultivation system in accordance with an embodiment of the invention.

While the invention is susceptible to various modifications and alternative forms, specifics thereof have been shown by way of example and drawings, and will be described in detail. It should be understood; however, that the invention is not limited to the particular embodiments described. On the contrary, the intention is to cover modifications, equivalents, and alternatives falling within the spirit and scope of the invention.

### DETAILED DESCRIPTION OF THE INVENTION

Algae cultivation systems can incorporate lighting systems in order to promote the growth of algae when ambient light from the sun is insufficient. By way of example, some algae cultivation systems can include lighting systems so that algae can grow even at night. In addition, some algae cultivation systems with relatively deep growth tanks can incorporate lighting systems so that light can be provided to areas of the tank besides just the surface.

However, the use of lighting systems can be problematic. By way of example, algae can have a tendency to adhere and grow on the surface of a light source. In the context of fixed position light sources, this can cause less of the light generated by the light source to actually pass into the cultivation tank over time as the adherent algae layer builds up and blocks the light. As such, efficiency of the cultivation system can decline over time.

The applicants have discovered that certain limitations of light sources for algae cultivation tanks can be overcome by effectively moving the light source through the cultivation tank. As such, embodiments described herein can include algae cultivation systems that include light sources that are configured so that they can be moved within the tank of an algae cultivation system. By way of example, a light source can be coupled to a rotor, which can be configured to be disposed within the tank of an algae cultivation system. As the rotor turns, the light source is moved through the tank.

The use of a light source mounted to a moveable component, such as a rotor, offers various advantages. First, turbulence on the surface of the light source as it moves through the liquid medium within the tank of a cultivation system reduces the rate at which adherent algae forms, thereby preserving efficiency of the system. Second, published data suggests that algae use light most efficiently when it is provided as a series of flashes of light. By mounting the light source to a component that moves within the liquid medium of the cultivation tank, a similar effect as a flashing light source can be provided because algae growing in the medium will periodically be in close contact with the light and then out of contact as the light source continues to move within the cultivation tank. Various aspects of exemplary embodiments will now be described in greater detail.

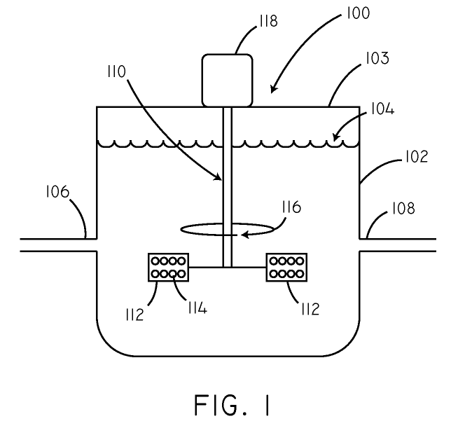

Referring now to FIG. 1, a schematic view of an algae cultivation system 100 is shown in accordance with an embodiment of the invention. The algae cultivation system 100 includes a tank 102. The tank 102 is configured to hold a liquid medium 104 which can contain algae. The tank 102 can be made of various materials including polymers, metals, cementitious materials, and the like.

The tank 102 can include an inflow conduit 106. By way of example, nutrients to support the growth of algae can be supplied through the inflow conduit. Gas containing carbon dioxide for fixation can also flow into the cultivation tank 102 through a gas-supply conduit (not shown). The gas can come from a source such as a power generation plant. The gas can include components other than just carbon dioxide. By way of example, the gas can also include nitrogen, carbon monoxide as well as various sulfur (SOₓ) and nitrogen (NOₓ) containing compounds.

The tank 102 can also include an outflow conduit 108. Algae for harvest and some of the spent liquid medium in which it grows can be removed from the cultivation tank 102 through the outflow conduit 108. The algae can be separated from the liquid, such as by gravity, centrifugation, or decanting, and then the liquid can be reconstituted and recycled. The algae can then be processed for various purposes. By way of example, in some embodiments, the algae can be processed in order to extract lipids which can then be processed in order to produce commercially valuable compositions. In some cases, the lipids can be subjected to catalytic esterification and transesterification reactions in order to produce an alkyl ester composition such as a biodiesel fuel composition. Exemplary techniques for producing alkyl ester compositions can be found in U.S. Publ. Pat. Appl. No. 2008/0051592, the content of which is herein incorporated by reference. In some cases protein from the algae can be extracted and used for feeding livestock. In some cases carbohydrates (such as cellulose) can be extracted from the algae, hydrolyzed, and then used in a fermentation process for the production of ethanol.

The tank 102 can be of various sizes from several gallons to several million gallons. In some embodiments, the depth of the tank 102 can be greater than about two feet. In some embodiments, the depth of the tank 102 can be greater than about five feet. A cover 103 can be disposed over the tank 102. The cover 103 can be made from a polymer, a glass, or crystal, amongst other materials. In some embodiments, the cover 103 can be removable to enable convenient access to the cultivation tank 104.

The system 100 can include a rotor 110. The rotor 110 can be configured to be disposed at least partially within the liquid medium 104. In this embodiment, the rotor 110 includes paddles 112 or blades. The rotor 110 is configured to be rotated as shown by arrow 116. The rotor 110 can rotate clockwise, counter-clockwise, or alternately in one direction and then the other. The rotor 110 can be driven by a drive unit 118, that can include, for example, an electric motor. Energy for the drive unit 118 can be provided in various ways such as through a connection to a local power grid, from an array of solar panels, or the like. The drive unit 118 can rotate the rotor 110 at various speeds as is desirable or optimal under the specific circumstances.

One or more light sources 114 can be coupled to the rotor 110. For example, one or more light sources 114 can be coupled to the paddles 112. The light sources 114 can emit electromagnetic radiation in the range of frequencies sufficient to support the growth of algae within the liquid medium 104. Various different types of light sources can be used. Exemplary light sources can includes, incandescent lamps, fluorescent lamps, fiber optic lighting sources, and light-emitting diodes. In a particular embodiment, the light sources 114 are light emitting diodes. As the rotor 110 rotates within the tank 102, the light sources 114 also rotate within the tank 102. As such, the light produced by the light source 114 is brought to different portions of the liquid medium 104.

In some embodiments, the light sources can be encapsulated with a translucent material, such as a glass or polymer, so that the surface that is in contact with the liquid medium of the tank is substantially smooth making adherence less likely.

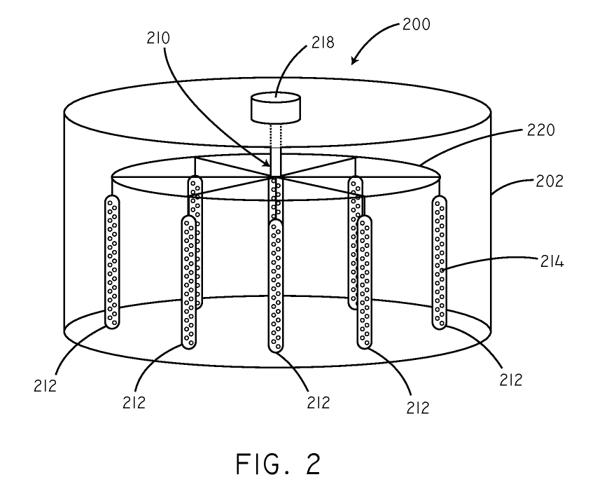

It will be appreciated that algae cultivation systems in accordance with embodiments herein can take on many different configurations. Referring now to FIG. 2, a schematic view of an algae cultivation system 200 is shown in accordance with another embodiment of the invention. The algae cultivation system 200 includes a rotor 210 that is configured to rotate within the tank 202. In this embodiments, the rotor 210 includes a frame 220 onto which a plurality of light bars 212 are coupled. Each of the light bars 212 can include one or more light sources 214. The rotor 210 is coupled to a drive unit 218 that causes the rotor 210 to rotate within the tank 202.

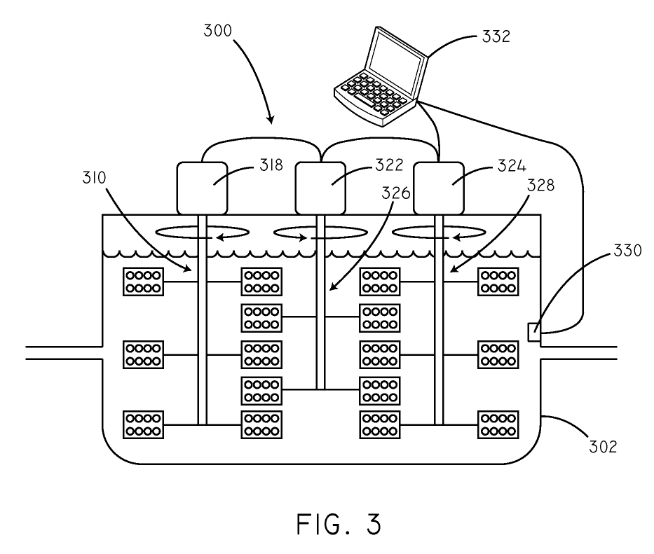

In some embodiments, algae cultivation systems can include multiple rotors. Referring now to FIG. 3, a schematic view of an algae cultivation system 300 is shown in accordance with another embodiment of the invention. The algae cultivation system 300 includes a first rotor 310, a second rotor 326, and a third rotor 328. The first rotor 310, second rotor 326, and third rotor 328 are coupled to a first drive unit 318, a second drive unit 322, and a third drive unit 325 respectively. The first rotor 310, second rotor 326, and third rotor 328 can each be configured to rotate at the same speed as the others or at different speeds. The first rotor 310, second rotor 326, and third rotor 328 can each be configured to rotate clockwise, counter-clockwise, or alternating between directions. In some embodiments, the first drive unit 318, second drive unit 322 and third drive unit 325 can each be in electrical communication with a control unit 332. The control unit 332 can be configured to control the first drive unit 318, second drive unit 322, and third drive unit 325. The control unit 332 can be located adjacent to the rest of the algae cultivation system or can be located at a remote location with communication taking place over a network, such as the Internet.

In some embodiments, the algae cultivation system 300 can include a sensor module 330. The sensor module 330 can be configured to measure various properties of the liquid medium and/or the algae in the tank 302. By way of example, the sensor module 330 can be configured to measure O₂ concentration, CO₂ concentration, pH, nitrogen concentration, temperature, light intensity, or the like. In some embodiments, the sensor module 330 can provide feedback to the control unit 332 that then used to determine aspects of rotor operation, such as rotation speed, light intensity of the light sources, rotation direction, and the like.

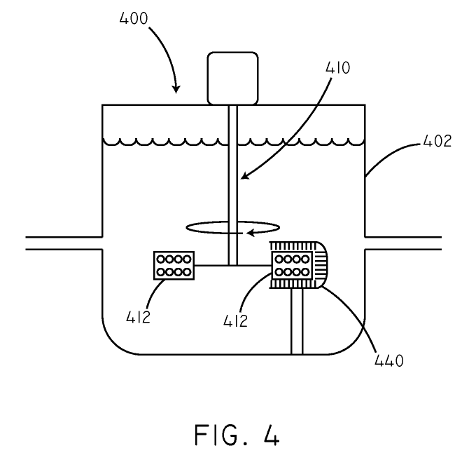

It will be appreciated that algae growth that is adherent to the light sources may reduce the effectiveness of the system. In some embodiments, algae cultivation systems can include features to limit algae growth on certain surfaces of the system, such as surfaces of the light source. For example, referring now to FIG. 4, an algae cultivation system 400 can include one or more brushes 440 configured so that the brushes contact elements of the rotor 410, such as the paddles 412, as they are rotated around within the tank 402. The brush 440 can act to remove algae which may adhere to a portion of the rotor 410 (such as the paddle 412), keeping the surface of the light sources clean. In some embodiments, the brush 440 can be configured to move between a first position where it contacts elements of the rotor 410 and a second position where it does not contact elements of the rotor 410.

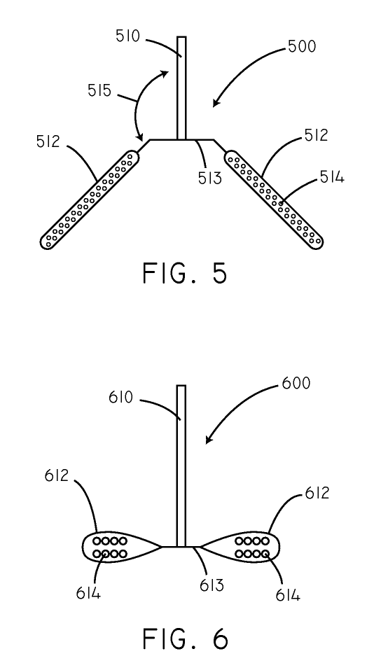

It will be appreciated that rotors including light sources in accordance with various embodiments herein can take on many different forms. Referring now to FIG. 5, a rotor 500 is shown in accordance with another embodiment of the invention. The rotor 500 can include a drive shaft 510 that is coupled to a cross-bracket 513. Light bars 512 are coupled to the cross-bracket 513 such that the angle 515 between the long axis of the light bars 512 and the main drive shaft 510 is approximately 135 degrees. However, it will be appreciated that many other specific angles are contemplated.

Referring now to FIG. 6, a rotor 600 is shown in accordance with yet another embodiment of the invention. The rotor 600 includes a drive shaft 610 that is coupled to a cross-bracket 613. Light bars 612 with a tear-drop shape, including light sources 614, are coupled to the cross-bracket 613.

Cultivation tanks used with embodiments herein can be constructed in various ways. In some embodiments, exemplary cultivation tanks can be constructed above-ground. However, in other embodiments, exemplary cultivation tanks are constructed such that at least a portion of the volume of the cultivation tank is below ground. While not intending to be bound by theory, it is believed that constructing cultivation tanks with at least a portion located below ground can be advantageous at least because the thermal mass of the earth can be used in order to help regulate the temperature of the contents of the cultivation tank. By way of example, in hotter months of the year the thermal mass of the earth may provide a net cooling effect to the contents of the cultivation tank and in colder months of the year the thermal mass of the earth may provide a net warming effect to the contents of the cultivation tank.

In some embodiments, an active heat control system can be used in order to regulate the temperature of the liquid medium inside of the cultivation tank. The heat control system can be configured to keep the temperature of the liquid medium within the cultivation tank within a desired range. By way of example, the heat control system can include a heating element, such as a resistive heating element, in order to generate heat. The heat control system can also be configured to cool the cultivation tank when desired. For example, the heat control system can be configured to actuate valves and/or a pump to add cooler liquid to the cultivation tank through an inflow conduit when desired. In some embodiments, an algae cultivation system can include a heat exchanger system. In some embodiments, heat can be added at the bottom of the cultivation tank and cooler components can be added to the top of the cultivation tank to aide in convection of the system.

The embodiments of the present invention described herein are not intended to be exhaustive or to limit the invention to the precise forms disclosed in the following detailed description. Rather, the embodiments are chosen and described so that others skilled in the art can appreciate and understand the principles and practices of the present invention. As such, it should be understood that many variations and modifications may be made while remaining within the spirit and scope of the invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, device, or other structure that is constructed or configured to perform a particular task or adopt a particular configuration. The phrase “configured” can be used interchangeably with other similar phrases such as arranged and configured, constructed and arranged, constructed, manufactured and arranged, and the like.

1. An algae cultivation system comprising:
a tank configured to hold a liquid medium;
a rotor disposed within the tank, the rotor configured to be rotated while disposed within the tank; and
a light source coupled to the rotor such that the light source rotates within the tank when the rotor rotates within the tank.

2. The algae cultivation system of claim 1, the light source comprising a plurality of light emitting diodes.

3. The algae cultivation system of claim 1, the rotor comprising one or more paddles.

4. The algae cultivation system of claim 3, the light source coupled to the paddles.

5. The algae cultivation system of claim 1, the light source embedded within a translucent material having a smooth exterior surface.

6. The algae cultivation system of claim 1, further comprising a drive unit coupled to the rotor.

7. The algae cultivation system of claim 6, the drive unit comprising an electric motor.

8. The algae cultivation system of claim 6, further comprising a control unit configured to control the drive unit.

9. The algae cultivation system of claim 6, further comprising a sensor configured to detect a parameter related to the growth of algae within the tank.

10. The algae cultivation system of claim 9, the control unit configured to control the drive unit based on data generated by the sensor.

11. The algae cultivation system of claim 1, further comprising a brush configured to intermittently contact the rotor.

12. The algae cultivation system of claim 1, comprising multiple rotors.

13. The algae cultivation system of claim 1, the tank having a depth of greater than about five feet.

14. The algae cultivation system of claim 1, further comprising a cover configured to fit over the top of the tank.

15. The algae cultivation system of claim 1, further comprising an inflow conduit in fluid communication with the tank and an outflow conduit in fluid communication with the tank.

16. A method of making an algae cultivation system comprising:
mounting a light source on a rotor; and
positioning the rotor within a tank configured to hold a liquid medium.

17. The method of claim 16, the light source comprising a light emitting diode.

18. A method of operating an algae cultivation system comprising:
filling a tank with a liquid medium including algae; and
rotating a light source mounted on a rotor within the tank.

19. The method of claim 18, the light source comprising a light emitting diode.
